(19) United States
(12) Patent Application Publication
(10) Pub. No.: US 2017/0156853 A1
(43) Pub. Date: Jun. 8, 2017

(54) TYMPANIC MEMBRANE REPAIR DEVICE

(71) Applicant: OTOLOGY SOLUTIONS LTD., JERUSALEM (IL)

(72) Inventors: MERRILL WEBER, OAK PARK, IL (US); BORIS FRENKEL, JERUSALEM (IL)

(21) Appl. No.: 15/325,113

(22) PCT Filed: Jul. 10, 2015
(86) PCT No.: PCT/IB15/55219

§ 371 Date: Jan. 10, 2017

Related U.S. Application Data
(60) Provisional application No. 62/022,743, filed on Jul. 10, 2014.

(51) Int. Cl. A61F 2/18; A61B 17/00

(52) U.S. Cl. CPC A61F 2/18; A61B 17/00491; A61F 2002/183; A61B 2017/00575; A61B 2017/00597; A61B 2017/0061; A61B 2017/00623

(57) ABSTRACT

A tympanic membrane repair device is disclosed which comprises a tube having a distal end having a patch expansion mechanism such as a balloon or mechanical expansion mechanism, wherein in a retracted configuration the patch expansion mechanism is positioned in close proximity to the tube wall and wherein in an expanded configuration the patch expansion mechanism is configured to at least partially extend away from the tube wall. The repair device is capable of delivering a grafting patch to cover a perforation on the underside of the tympanic membrane. A method of using the device is also disclosed.

### FIELD OF THE INVENTION

This disclosure relates to a tympanic membrane repair device and more particularly to a device and method for delivering a patch to a perforated tympanic membrane and attaching the patch to repair the perforated tympanic membrane.

### BACKGROUND OF THE INVENTION

The tympanic membrane, or the eardrum, is a layer of tissue that separates the middle ear and the outer ear canal. The tympanic membrane receives sound pressure waves and transmits the waves to the middle ear. The middle ear is mainly responsible for sound detection and balance. The middle ear contains bones that transmit sound pressure waves to the inner ear and then to the brain via the auditory nerve. Thetympanic membrane can rupture or become perforated in various ways. Some ways include a blast injury, air travel, a sports injury, and infection. Although a perforated tympanic membrane can heal spontaneously, there are some situations in which medical intervention is required. In view of this, some methods and devices have been developed to repair a perforated tympanic membrane.

Tympanoplasty is a procedure in which the perforated eardrum is patched with a graft material such as muscle fascia. The patch may be applied by using techniques including an overlay technique or an underlay technique. In the overlay technique, the patch remains on the outer surface of the tympanic membrane, covering the perforation from the outside (i.e. the side facing the exit of ear canal). In the underlay technique, the patch is delivered through the perforation, through a slit made elsewhere in the tympanic membrane, or through a surgical opening created in the outside of the ear. The underlay procedure may be the preferred method due to certain clinical advantages.

Some physicians or surgeons (providers) may utilize a fat plug myringoplasty procedure in which fat is harvested from the patient and is inserted through the debrided perforation. As a variant, a hyaluronic acid fat graft myringoplasty procedure may be employed in which a patient is administered a local anesthetic, fat is then harvested from behind the patient's ear, a bioresorbable sponge of hydrogel or similar material is pushed through the perforation to pack the middle ear, and then the fat is laid on top of the sponge to allow the fat to bulge through the perforation. Care is taken so that the fat does not bulge too high above the perforation. Once the fat is properly positioned in place, one or more hyaluronic acid ester patches are laid on top of the perforation to overlap the epithelium edge around the perforation. The patches are then covered with another sponge that has been soaked in saline to prevent their displacement. The ear canal is then filled with an ointment such as bacitracin/polymyxin. An example of a sponge used in this procedure is manufactured by Pfizer under the trademark Gelfoam®. Also, an example of a patch that may be used is made by Medtronic Xomed under the trademark EpiDisc®.

In an “over-under myringoplasty”, the provider places a graft behind the remnants of the tympanic membrane, but passes the graft on top of the handle of the malleus, one of the components of the tympanic membrane. This is done in an attempt to avoid atelectasis of the middle ear.

Another procedure, known as “simple underlay myringoplasty” (SUM), was introduced in Japan in 1989. It has become a common procedure in Japan for treating perforated eardrums. In performing a SUM, the tympanic membrane is anesthetized. Then, the margin of the perforation is cut and removed with a fine pick through an ear speculum. It is also known to use a curette, or another debriding device that is inserted through the perforation in the tympanic membrane to clean or scrape the edge of the perforation before a patch is put in place in order to make a vascular bed for grafting. Subcutaneous tissue or temporalis fascia is used for the graft and is trimmed to twice the diameter of the perforation, and then the center of the remaining graft tissue is marked with a marker pen. The graft is stretched in the middle-ear cleft, placed through the center of the perforation into the middle ear, and then lifted against the inner side of the perforation with its previously-marked center placed at the center of the perforation. After the provider checks to verify that the graft continues to make contact with the margin of the perforation, a few drops of an adhesive material such as fibrin glue are applied at the contact area. No packing is deemed necessary in a SUM. A great deal of manual dexterity is required on the part of the provider.

Although these various procedures have been used to repair a perforated tympanic membrane, there is still a need for a minimally invasive procedure or device to be used to perform the procedure. Therefore, it would be desirable to have a tympanic membrane repair device that is capable of placing a patch on the underside of the tympanic membrane to adhere the patch in place for repair of a perforation in the tympanic membrane. It would also be desirable to provide a tympanic membrane repair device that does not require extensive skill or experience in order to position the patch on the underside of the tympanic membrane. It would also be advantageous to have a tympanic membrane repair device that can easily position the patch on the underside of the perforation of the tympanic membrane, and which can be removed easily once the patch is in position. The devices and methods described herein provide advantages over known procedures, such as SUM, in that the patch is delivered by means of a simple device that holds it in direct contact with the perforation, and optionally, the present invention may not require application of an adhesive material to affix the patch in place. The devices and methods described herein do not require special dexterity, and render the procedure faster, more controlled, and more predictable. Moreover, it can be performed with one hand, which enables the provider to hold an endoscope, otoscope, microscope, or other visioning device, with the other hand in order to view the procedure.

### SUMMARY OF THE INVENTION

There is provided, in accordance with one embodiment of the present invention, a tympanic membrane repair device. The device includes a tube having a tube proximal end, a tube distal end and a tube wall extending from the tube proximal end to the tube distal end along a longitudinal axis, and a patch expansion mechanism positioned adjacent to the tube, the patch expansion mechanism having a retracted configuration and an expanded configuration, wherein in the retracted configuration, the patch expansion mechanism is positioned in close proximity to the tube wall and wherein in the expanded configuration, the patch expansion mechanism is configured to at least partially extend away from the tube wall at an angle to the longitudinal axis. The angle may be, for example 90 degrees or less.

In accordance with further features in embodiments of the present invention, the patch expansion mechanism may be positioned external to or internal to the tube wall. In some embodiments, the patch expansion mechanism includes a sliding mechanism configured to move in a proximal and distal direction along the longitudinal axis, and may further include a patch expansion member having a proximal end and a distal end, wherein in the retracted configuration, the expansion member proximal end is held by the sliding mechanism such that the patch expansion member is substantially aligned with the longitudinal axis, and wherein upon movement of the sliding mechanism, the patch expansion member is configured to extend outwardly with respect to the distal end of the tube wall. In some embodiments, the sliding mechanism includes a patch support ring surrounding the patch expansion member and a patch support ring retraction mechanism attached to the patch support ring, wherein movement of the sliding mechanism includes proximal movement of the patch support ring retraction mechanism, resulting in release of the patch expansion member from the patch support ring. In other embodiments, the sliding mechanism includes a runner positioned coaxially with respect to the tube wall, where the expansion member proximal end is attached to the runner, and a stop member at the tube distal end, wherein the movement of the sliding mechanism includes distal movement of the runner until the runner reaches the stop member, resulting in distal and outwardly extending movement of the patch expansion member proximal end.

In accordance with further features in embodiments of the present invention, the device may further include a distal patch holder at the tube distal end, wherein in some embodiments, the distal patch holder is a curved hook. In some embodiments, the distal patch holder is a straight hook. In additional embodiments, the distal patch holder is a set of bristles, held in place by a removable cap. In these embodiments, a patch positioned on the device may have an opening or a perforation through which the distal patch holder is positioned.

In accordance with additional features of the present invention, the patch expansion mechanism is an expandable balloon positioned inside the tube. The expandable balloon may have, for example, a toroidal shape and be placed perpendicular to the longitudinal direction of the tube, such that upon expansion of the balloon, a repair patch positioned thereon will extend into a substantially flat shape with outer edges positioned away from the tube. In some embodiments, a sliding mechanism may be attached to the expandable balloon to push the expandable balloon distally through an opening in the tube. In some embodiments, an inflation channel may extend from the balloon to the tube proximal end, and may be configured to provide liquid or gas inflation fluid into the expandable balloon.

In accordance with additional features of the present invention, the device may further include a repair patch, having a flexible surface with outer edges, wherein the repair patch is held by the patch expansion mechanism, wherein in the retracted configuration, the flexible surface is folded into an umbrella-like or optionally a cone-like shape wherein the outer edges are in close proximity to the tube wall and in the expanded configuration, the flexible surface assumes a substantially flat configuration wherein the outer edges are extended away from the tube wall. In some embodiments, a removable or degradable cord is attached to a proximal surface of the repair patch, wherein pulling the cord proximally causes the repair patch to flatten against the tympanic membrane from inside the ear, until the repair patch is firmly in place.

In accordance with additional features, the tube may further include an input port connected to various output ports through an interior lumen formed in the tube.

There is provided, in accordance with one embodiment of the present invention, a method for repairing a perforation in a tympanic membrane of an ear. The method includes providing a repair device having a tube with a proximal end, a distal end, and a tube wall extending from the proximal end to the distal end along a longitudinal axis, and a patch expansion mechanism having a retracted configuration and an expanded configuration, providing a repair patch held by the patch expansion mechanism in the retracted configuration, such that outer edges of the repair patch are in close proximity to the tube wall, inserting the repair device into an ear canal and through a perforation in a tympanic membrane of the ear, expanding the patch by operation of the patch expansion mechanism, such that the outer edges of the repair patch are extended away from the tube wall, adhering the patch to the underside of the tympanic membrane, retracting the patch expansion mechanism, and removing the repair device from the ear canal. In some embodiments the perforation may be debrided prior to inserting the repair device into the ear canal. In some embodiments, the tympanic membrane is anesthetized prior to inserting the repair device into the ear canal.

In accordance with additional features in embodiments of the present invention, the method may further include inserting an adhesive material into the ear canal for adhering the repair patch to the tympanic membrane. This may be done via an interior lumen of the tube, or by means of a separate adhesive applicator. In some embodiments, the repair patch is included on the repair device, and in other embodiments, the repair patch is added to the repair device by the provider or other medical professional prior to use. The method may further include holding the patch in place against the underside of the tympanic membrane until it is secure. In some embodiments, the patch expansion mechanism is a balloon which is expanded via an inflation lumen.

The present disclosure provides a tympanic membrane repair device that is capable of delivering a patch through a perforation in an eardrum and securing the patch to the eardrum.

The present disclosure provides a tympanic membrane repair device that is small, lightweight, easy to handle, and easy to operate.

The present disclosure also provides a tympanic membrane repair device which can be easily employed with highly reliable results in repairing a perforation in the tympanic membrane.

The present disclosure is related to a tympanic membrane repair device that does not require extensive skill or experience of the provider to position the patch on the underside of the tympanic membrane during a repair procedure.

The present disclosure further provides a tympanic membrane repair device that can be employed to easily position the patch on the underside of the perforation of the tympanic membrane and remove the device once the patch is in position.

The present disclosure provides a tympanic membrane repair device that is an alternative to invasive surgical procedures that are used to repair a perforation in the tympanic membrane.

The present disclosure is also directed to a tympanic membrane repair device that initially has a patch in a collapsed position to insert the patch through a perforation in the tympanic membrane and then expands the patch to position the patch on the underside of the tympanic membrane for adhering the patch to the tympanic membrane.

These and other advantages of the present disclosure will become apparent after considering the following detailed specification in conjunction with the accompanying drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIG. 1 is a partial perspective view of a tympanic membrane repair device constructed according to the present disclosure;

FIG. 2 is a partial perspective view of the tympanic membrane repair device shown in FIG. 1 having a grafting patch positioned on the device in a collapsed position;

FIG. 3 is a partial perspective view of the tympanic membrane repair device shown in FIG. 2 having the grafting patch positioned on the device in an expanded position;

FIG. 4 is a schematic view of the tympanic membrane repair device being inserted into an ear canal;

FIG. 5 is a schematic view of the tympanic membrane repair device being inserted through a perforation in a tympanic membrane of an ear and a patch attached to the device being in an open position;

FIG. 6 is a schematic view of a tympanic membrane of an ear having a patch adhered over a perforation in the tympanic membrane;

FIG. 7 is a perspective view of another embodiment of a tympanic membrane repair device constructed according to the present disclosure;

FIG. 8 is a partial perspective view of the tympanic membrane repair device shown in FIG. 7 having a grafting patch positioned on the device in a collapsed position;

FIG. 9 is a partial perspective view of the tympanic membrane repair device shown in FIG. 8 having the grafting patch positioned on the device in an expanded position;

FIG. 10 is a partial perspective view of yet another preferred embodiment of a tympanic membrane repair device constructed according to the present disclosure;

FIG. 11 is a partial perspective view of the tympanic membrane repair device shown in FIG. 10 in a deployed state;

FIG. 12 is a partial perspective view of another preferred embodiment of a tympanic membrane repair device constructed according to the present disclosure;

FIGS. 13A and 13B are perspective views of another preferred embodiment of a tympanic membrane repair device constructed according to the present disclosure, shown in a first position and a second position, respectively;

FIG. 13C is a transverse cross-sectional illustration of FIG. 13A, in accordance with embodiments of the present invention;

FIG. 14 is an illustration of the device of FIGS. 13A-13B in an expanded position;

FIG. 15 is an illustration of an adhesive applicator, in accordance with embodiments of the present invention; and

FIG. 16 is a flow chart diagram illustration of the steps of a method of repairing a tympanic membrane, in accordance with embodiments of the present invention.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION OF THE INVENTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

The present invention is directed to a tympanic membrane repair device and methods of use thereof. The principles and operation of systems and methods according to the present invention may be better understood with reference to the drawings and accompanying descriptions.

Before explaining at least one embodiment of the present invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

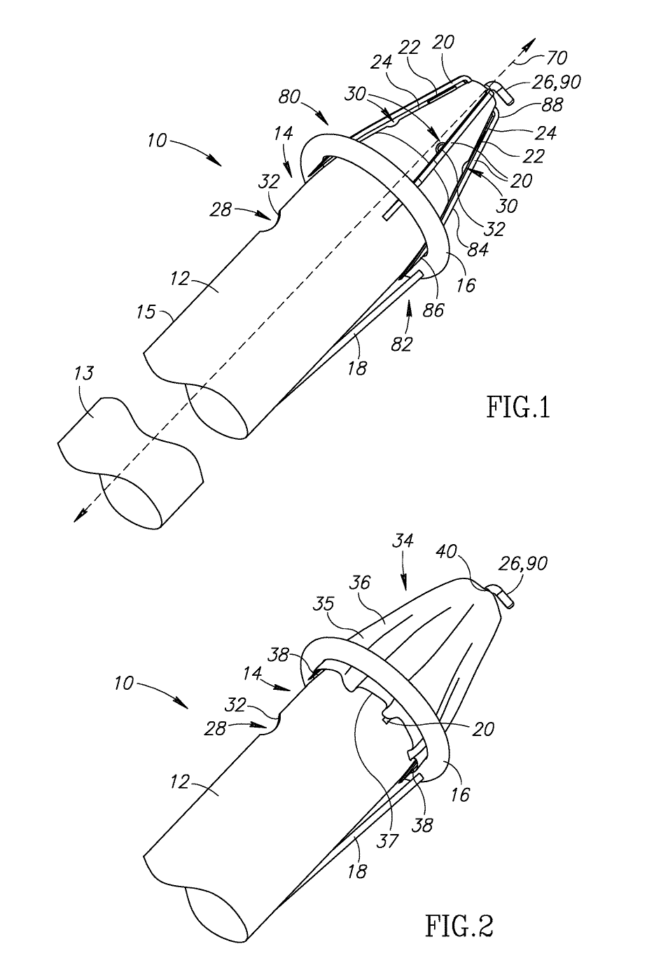

Referring now to the drawings, wherein like numbers refer to like items, number 10 identifies a tympanic membrane repair device constructed according to the present disclosure. With reference now to FIG. 1, the tympanic membrane repair device 10 comprises a tube 12 having a tube proximal end 13, a tube distal end 14, and a tube wall 15 extending from tube proximal end 13 to tube distal end 14 along a longitudinal axis 70. The device 10 includes a patch expansion mechanism 80 for expanding a patch placed thereon, as will be described further hereinbelow. In the embodiment shown in FIG. 1, patch expansion mechanism 80 includes a sliding mechanism 82 and at least one patch expansion member 84 having an expansion member proximal end 86 and an expansion member distal end 88. In the embodiment shown herein, sliding mechanism 82 includes a patch support ring 16 and a patch support ring retraction mechanism 18. Patch expansion member 84 includes patch expansion struts 20, patch expansion struts deployment mechanisms 22 and patch expansion struts deployment mechanisms slits 24. Device 10 further includes a distal patch holder 90, which in the embodiment shown in FIG. 1 is a patch securing hook 26. The tube 12 has an input port 28 that is connected to various output ports 30 through an interior lumen 32 formed in the tube 12. The input port 28, the output ports 30, and the lumen 32 are used to deliver an adhesive material such as fibrin glue, as will be explained further herein. The tympanic membrane repair device 10 is shown in a collapsed or retracted position. The tympanic membrane repair device 10 may be positioned or moved into an expanded position, as will be explained in further detail herein. Further, the tube 12 may be hollow to house various other mechanisms or components. The patch support ring 16 may be retracted by use of the patch support ring retraction mechanism 18. The patch expansion struts 20 may be held in place by the patch support ring 16. When the ring 16 is retracted by the mechanism 18, the struts 20 are free to move away from the tube 12 by use of the patch expansion struts deployment mechanisms 22.

FIG. 2 illustrates a partial perspective view of the tympanic membrane repair device 10 having a grafting patch 34 positioned on the device 10. The grafting patch 34 has a flexible surface 35, including a distal surface 36 and a proximal surface 38. The grafting patch 34, when expanded, has a flattened disk-like shape with an outer edge 37 around the periphery of the patch 34. When the grafting patch 34 is positioned on the device 10, as shown in FIG. 2, the distal surface 36 is visible (i.e. it is on the outside) while the proximal surface 38 is not generally visible (i.e. it is on the inside since the patch 34 is folded over the distal end 14 of the device 10). The patch 34 may further include an opening or perforation 40. The opening 40 allows the hook 26 to be inserted therethrough to secure the patch 34 to the device 10. The patch 34 is placed over and against the patch expansion struts 20, the patch expansion struts deployment mechanisms 22, and the patch expansion struts deployment mechanisms slits 24, all of which are hidden in this particular view by the patch 34. The patch 34 is positioned, crimped, or tucked underneath the patch support ring 16. In this manner, the patch 34 is held in place on the device 10 in a collapsed or retracted position. In this position, the patch 34 may resemble a folded umbrella, or a cone shape, wherein edges 37 of the patch 34 are in close proximity to tube wall 15. The edges 37 have folds and creases which will be straightened out upon deployment. Although the struts 20 are shown extending out from the patch 34, it is possible and contemplated that the patch 34 may extend over struts 20. In some embodiments, the patch 34 may have a hem or pockets incorporated into the proximal surface 38 and the struts 20 may be inserted into the hem or pockets to further secure or hold the patch 34 in place.

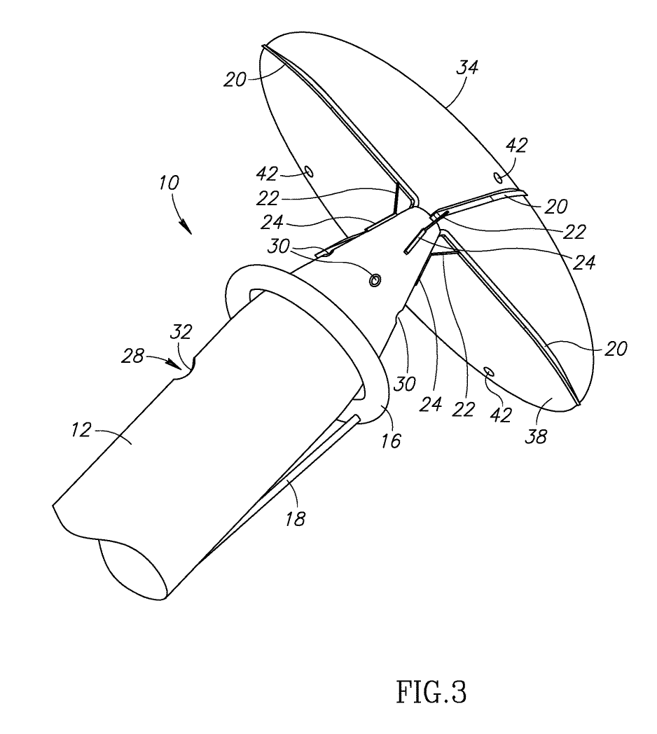

Referring now to FIG. 3, the device 10 is shown in an expanded or unfurled position. The proximal surface 38 of the patch 34 is depicted having drops of adhesive material 42 on the surface 38. The drops of adhesive material 42 are positioned adjacent to the output ports 30. The patch expansion struts 20 and the patch expansion struts deployment mechanisms 22 are in their extended positions to extend the patch 34 outwardly away from the device 10. The patch expansion struts deployment mechanisms 22 are extended out of the slits 24. In this expanded position or orientation, the patch 34 may be adhered to a perforation formed in a tympanic membrane. The patch 34 has also been moved away from being tucked underneath the patch support ring 16. Although not shown, the hook 26 is still holding the patch 34 in place. Further, it is possible and contemplated that the hook 26 may be retractable to release the patch 34. Various mechanisms used to operate or actuate the patch support ring retractable mechanism 18, the patch expansion struts deployment mechanisms 22, and the hook 26 may be positioned within the tube 12. Controls (not shown) may extend out of a proximal end of the device 10.

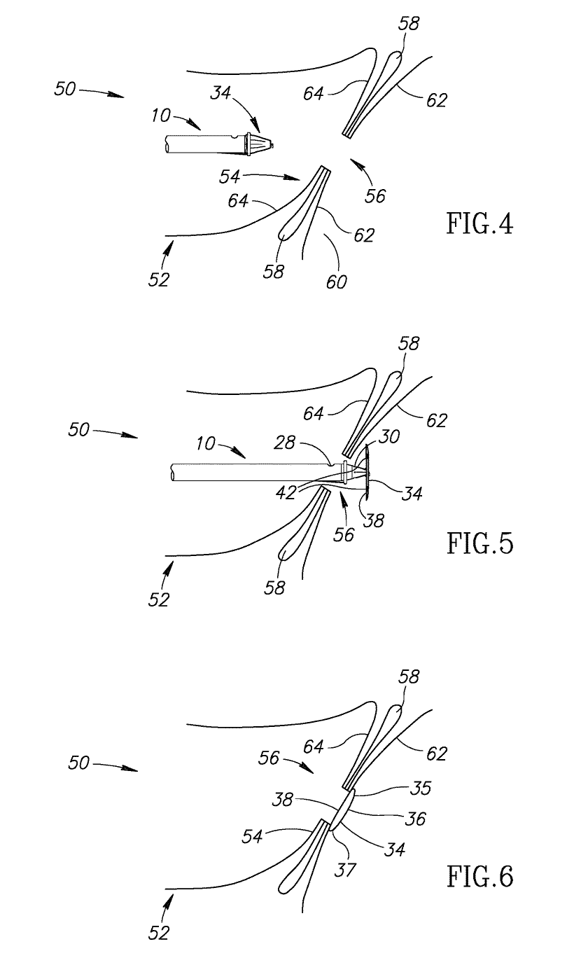

FIG. 4 depicts the tympanic membrane repair device 10 being inserted into an ear canal 50 of an ear 52. A tympanic membrane 54 having a debrided perforation 56 is shown in cross-section. The tympanic membrane 54 consists of other elements such as a lamina propria 58, a mucosal layer 60, an underlay side 62, and overlay side 64. The device 10 is small enough in dimension to be inserted into the ear canal 50 and through the debrided perforation 56. For example, an outer diameter of device 10 may be in a range of less than 10 mm, and may be 4-5 mm in diameter or less. The tympanic membrane repair device 10 is also shown having the patch 34 in place with both the device 10 and the patch 34 in a collapsed state ready to be opened or expanded.

Referring now in particular to FIG. 5, the repair device 10 has been inserted through the perforation 56 and the device 10 has been actuated to expand the patch 34. Prior to expanding the patch 34, an adhesive material 42, which may be, for example, fibrin glue, has been inserted through the inlet port 28 to flow out through the outlet ports 30 to deposit drops of adhesive material 42 on the proximal surface 38 of the patch 34. The adhesive material 42 will be used to adhere the patch 34 in place to the underlay side 62 of the tympanic membrane 54. As can be appreciated, only a small amount of adhesive material is needed. However, there may be circumstances in which more adhesive material may be needed depending on the size of the perforation 56. In some embodiments, no adhesive material is applied at all, as the periphery or edge of the patch 34 is made of or coated with a biomaterial that becomes adhesive when it comes into contact with the vascular bed that is created by debridement of the perforation in the tympanic membrane. The patch expansion struts 20 and the patch expansion struts deployment mechanisms 22 are expanded to open up the patch 34 for attachment to the underlay side 62.

FIG. 6 illustrates the proximal surface 38 of the patch 34 being adhered to the underlay side 62 of the tympanic membrane 54. The patch 34 is used to close the perforation 56 in an effort to heal the perforation 56. The patch 34 may be made of a biocompatible material or polymer. Some examples of material that the patch 34 may be made of are temporalis fascia or fat harvested or extracted from behind a patient's ear, hyaluronic acid, perichondria, rolling paper, rice paper, silk fibroin membranes, micropore tape, atero-collagen, cellophane sheets, polyglycolic acid sheets, EpiDisk®, Alloderm® (an allograft product made from cadaverous tissue by LifeCell), or other bovine, porcine, or non-organically derived materials. In accordance with embodiments of the present invention, the patch 34 is bioresorbable. As epithelial cells grow over the patch 34, it is contemplated that the patch 34 will degrade and the perforation 56 will heal. The patch 34 may be of different dimensions, diameters, and configurations. The patch 34 includes a flexible surface 35 as well as an outer edge 37 around the periphery of the patch 34. By way of example only, the patch 34 may be round, disc, oval, or circular in shape. The patch 34 may have a relatively thin width from distal surface 36 to proximal surface 38, such as, for example, 1 mm or less. The patch 34 may have a diameter ranging from 1 mm to 10 mm. It is desired that the patch 34 be larger than the perforation 56 so that the patch 34 can cover the perforation 56 and be affixed to the debrided edges of the perforation 56. For example, the diameter of the patch 34 may be at least 2 mm greater that the diameter of the perforation 56 to be covered. It is also contemplated that more than one patch 34 may be used with the device 10. The device 10 may also be constructed of any suitable material such as polymer, plastic, metal, or stainless steel.

In operation of the tympanic membrane repair device 10, a provider or other medical professional may locate the perforation 56 and determine the size of the perforation 56. Depending on the size of the perforation 56, a patch 34 having a certain diameter and shape may be selected to be placed on the device 10. A debridement procedure may be performed on the tissue that surrounds the perforation 56. The patch 34 is placed on the distal end 14 of the repair device 10. Alternatively, the patch 34 is placed on the distal end 14 of the repair device 10 by the manufacturer and delivered to the provider already positioned, crimped, tucked under the patch support ring 16, or otherwise affixed on the repair device 10. The device 10 and the patch 34 are inserted into the ear canal 50 and through the perforation 56. Alternatively, only the patch 34 and the patch expansion mechanism 80 are passed through the perforation 56 while the device 10 remains in the ear canal. Once the patch 34 and the device 10 and/or patch expansion mechanism 80 are passed through the perforation 56, adhesive material may be inserted into the inlet port 28 to pass through the outlet ports 30 and onto the proximal surface 38 of the patch 34. Alternatively, adhesive material may be inserted prior to insertion of the patch 34 and the device and/or patch expansion mechanism 80 passing through the perforation 56. This may be done, for example, using a separate adhesive applicator, as will be described in further detail hereinbelow. Alternatively, it may not be necessary for any adhesive material to be separately applied, as disclosed supra. The patch expansion mechanism 80 is then actuated to expand the patch 34 and the device 10 and/or patch expansion mechanism 80 is moved back toward the tympanic membrane 54 to adhere the patch 34 to the underlay surface 62 of the tympanic membrane 54. Movement of the patch 34 proximally toward the tympanic membrane 54 may be done manually or via a mechanism such as a cord enclosure 420 having a ratcheting means as will be described hereinbelow. The device 10 may be held in place for a sufficient time for the adhesive material 42 to adhere the patch 34 to the surface 62. Once a sufficient time has elapsed, the provider may operate the device 10 to retract the patch expansion struts deployment mechanisms 22 and the hook 26 in order to fully release the patch 34 from the device 10. The device 10 is then removed from the ear canal 50 and the ear 52.

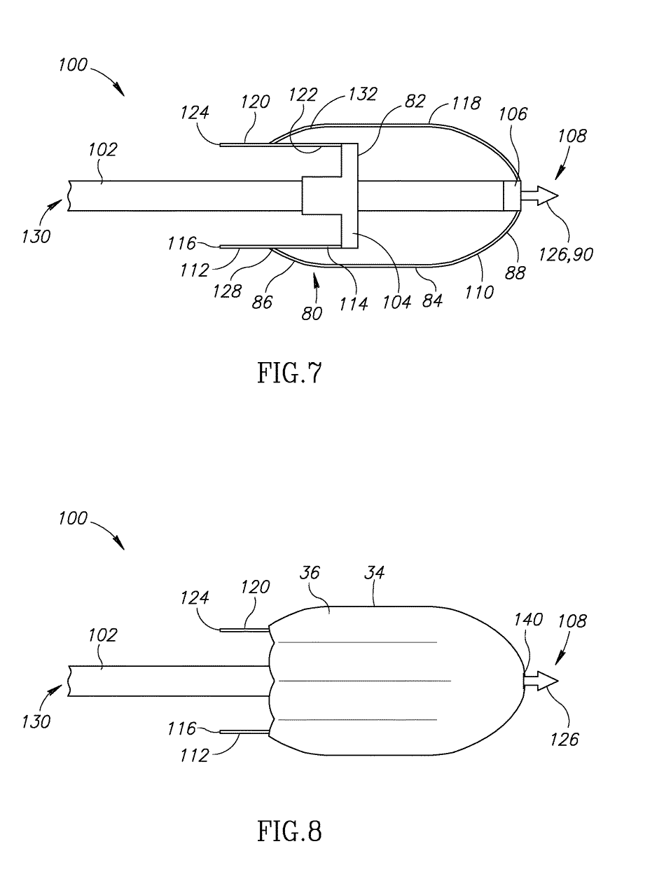

FIG. 7 depicts another embodiment of a tympanic membrane repair device 100 constructed according to the present disclosure. The tympanic membrane repair device 100 comprises a tube 102 having a runner 104 that is capable of sliding coaxially along the tube 102. The tube 102 may be hollow for housing various mechanisms or lumens (not shown). In the embodiment shown herein, patch expansion mechanism 80 includes sliding mechanism 82 which includes runner 104 and a stop member 106 which prevents the runner 104 from falling off the tube 102 at a distal end 108. Patch expansion mechanism 80 further includes a patch expansion member 84 having an expansion member proximal end 86 and expansion member distal end 88, wherein expansion member includes a first rib 110 connected to the stopper member 106 and to a first extended member 112. The first extended member 112 has a first end 114 connected to the runner 104 and a second free end 116. A second rib 118, opposite first rib 110, is connected to the stopper member 106 and a second extended member 120. The second extended member 120 has a first end 122 connected to the runner 104 and a second free end 124. Distal patch holder 90 includes a retractable hook or barb 126 positioned at the distal end 108. As can be appreciated, a patch (not shown) may be placed on the device 100 and through the barb 126. The device 100 is shown in its initial or retracted position. The repair device 100 also has an outlet port 128 formed in the first rib 110 for expelling adhesive material that is inserted into the device 100 through a proximal end 130. The second rib 118 also has an outlet port 132 for allowing adhesive material to flow out of the port 132.

With particular reference now to FIG. 8, a patch 34 has been placed over the device 100. The patch 34 has a distal surface 36 having an opening 140 through which the barb 126 may pass. The opening 140 is very small in size, on the order of, for example, less than 1 mm and in some embodiments, less than 0.1 mm. The device 100 is shown in a retracted position and the stretchers 112 and 120 are parallel to the tube 102. The device 100 is in an umbrella-like closed orientation. In this position or orientation, a provider may pass the device 100 and/or patch expansion mechanism 80 into an ear canal and through a perforation in a tympanic member of an ear.

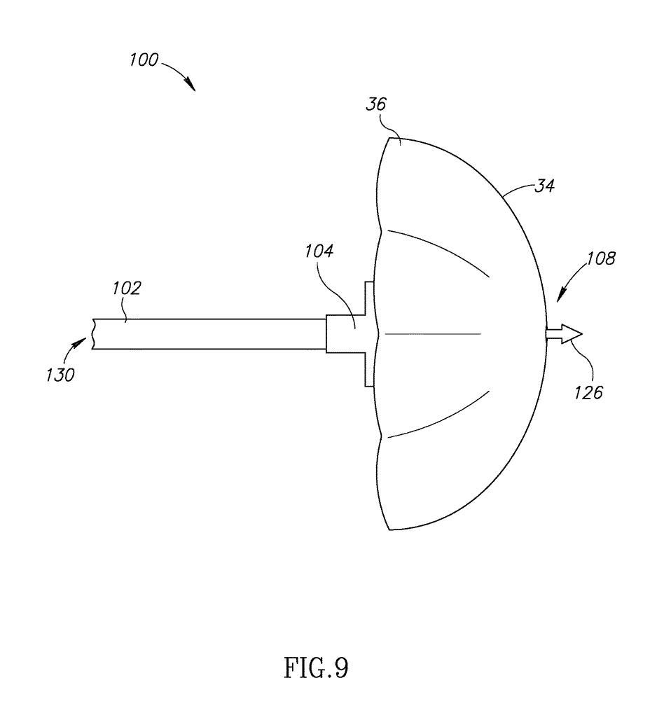

FIG. 9 illustrates the device 100 in a partially opened position. The patch 34 has been partially spread out and is almost ready to be adhered to an underlay surface of a tympanic membrane. The runner 104 has been moved up the tube 102 toward the distal end 108 of the device 100. The runner 104 can be moved all the way up the tube 102 until the stopper member 106 is encountered. Various mechanisms not shown may be provided at the proximal end 130 and used to retract the device 100 once the patch 34 has been positioned over a perforation in a tympanic membrane. Also, adhesive material may be inserted at the proximal end 130, or at another point along the tube wall, to flow through the tube 102. The device 100 is in an umbrella-like partially open position. The device 100 functions like an umbrella to first contain the patch 34 in a closed position, then to open the patch 34, release the patch 34, and finally to retract the device 100 once the patch 34 is released. In this manner, the device 100 may be used to deliver the patch 34 through a perforation in a tympanic membrane to close a perforation formed in the tympanic membrane.

Other alternative devices are possible and contemplated. In particular, an inner ring that is positioned between the patch 34 and the struts 20, on one hand, and the tube 12, on the other hand, and is actuated by pushing the ring laterally toward the distal end 14 of the repair device 10 and away from the tube 12, may be employed. There can be two rings in this particular embodiment, the ring just discussed and the patch support ring 16. It is also possible that only one ring will be required to actuate the device 10. In an alternative one-ring embodiment, a provider will retract the patch support ring 16 so that the patch support ring 16 is proximal to the patch 34 and the proximal ends of the struts 20 will partially extend away from the tube 12 of the device 10. The ring 16 may be pushed forward laterally along the device 10, thereby forcing the proximal ends of the struts 20 to angle outward further until the patch 34 is substantially or fully extended. The adhesive material can also be stored in an adhesive material deployment sub-assembly housed within the tube 12 or a provider or other medical professional may dab a few drops of adhesive material onto the periphery of the patch 34 before inserting the device 10 into the ear canal and the patch expansion mechanism 80 through the perforation. Alternatively, an adhesive applicator may be used to apply adhesive material directly to a surface of the tympanic membrane. Alternatively, adhesive material may be applied to the proximal surface 38 of the patch 34 at one or several contact points with a border of the perforation. Alternatively, no adhesive material may be required to be applied to the patch 34 due to its composition as described supra. It is also possible that the patch 34 may be held in place against the device 10 by use of suction, adhesion, or other means that may not require perforation of the patch.

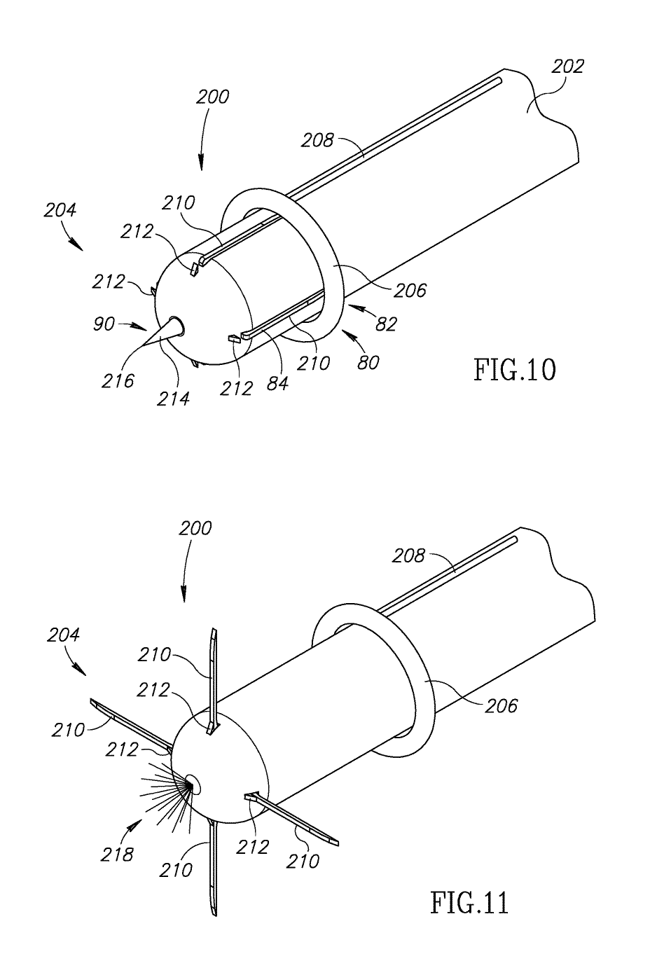

FIG. 10 is a partial perspective of another preferred embodiment of a tympanic membrane repair device 200 constructed according to the present disclosure. The tympanic membrane repair device 200 comprises a tube 202 having a distal end 204 having a patch expansion mechanism 80 including a sliding mechanism 82 which includes a patch support ring 206 and one or more patch support ring retraction mechanisms 208. The patch expansion mechanism 80 further includes one or multiple patch expansion members 84, which include patch expansion struts 210 and lips 212. Device 200 further includes a distal patch holder 90 which includes a removable cap 214 that has a point 216 at a distal tip thereof. A patch (not shown) may be placed through the point 216 and crimped below the patch support ring 206. The cap 214 may be removed and the patch will be held in place, as will be described further herein. Although not shown in detail, the device 200 may comprise various other components or elements which have been discussed above in this specification. By way of example only, the device 200 may include an input port and an output port for use of an adhesive material or a deployment mechanism or mechanisms for expanding and retracting the patch expansion struts 210. Further, although a pair of retraction mechanisms 208 are shown, it is possible to employ more than two of the retraction mechanisms or just one retraction mechanism 208.

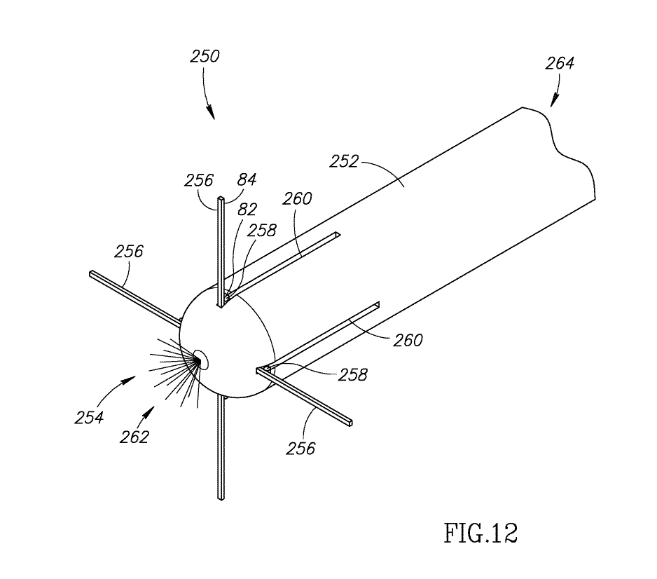

Referring now to FIG. 11, the cap 216 has been removed from the device 200 to reveal a plurality of bristles 218. The bristles 218 are used to hold a patch (not shown) in place. The bristles 218 spread out laterally once the cap 216 is removed. The patch support ring 206 has also been retracted in this particular view by use of the patch support ring retraction mechanism 208. The patch expansion struts 210 are also shown in an extended or open position. In this manner, a patch that has been attached to the device will be opened or fanned out for attachment to the tympanic membrane, as has been discussed previously herein. Although bristles 218 are shown, it is also contemplated that various other structures which can expand, unfurl, or open up made be used so that a patch is held in place.

FIG. 12 illustrates another embodiment of a tympanic membrane repair device 250 constructed according to the present disclosure. The tympanic membrane repair device 250 comprises a tube 252 having a distal end 254, patch expansion struts 256 (i.e. patch expansion members 84) each having a roller assembly 258 (i.e. sliding mechanism 82) that is used to expand or retract the struts 256, strut slits 260 into which each strut 256 may collapse, and a plurality of bristles 262. Again, other structures that have been previously described above may be incorporated into the device 250. The struts 256 are designed to spread out laterally to fan open a patch (not shown) that is connected to the device 250. Once the struts 256 are spread out and the patch is deployed, the struts 256 may be pulled back into the slits 260 by either rolling the struts 256 up inside or by pulling the struts 256 toward a proximal end 264 of the device 250. Due to the roller assemblies 258 within the tube 252, the struts 256 will extend at an angle from the tube 252, and can extend up to a 90 degree angle from the tube 252. When the struts 256 are retracted into the tube 252, they enter slits 260.

The devices 200 and 250 may be operated in the following ways. A patch is perforated in the middle by the cap 216. The cap 216 may be removed to expose the plurality of bristles 218 or 262. The patch may be pulled downward to crimp the patch around the tube 202 or 252 and underneath the ring 206. This can pull the struts 210 or 256 in toward the tube 202 or 252, respectively. Alternatively, a patch-flattening mechanism may be affixed to the center of the patch, either pre-loaded or by the provider performing the procedure or another medical professional. The ring 206 encases the patch and the struts 202 and 252 to hold the patch in place. Adhesive material may be delivered though an interior lumen or canal within the device 200 or 250. Alternatively, adhesive material may be dabbed onto the patch before or after the patch is crimped. Alternatively, it may not be necessary to apply adhesive to the patch at the time of the procedure due to the composition of the patch, as disclosed supra. The distal end 204 or 254 of the device 200, 250 and/or patch expansion mechanism 80 is delivered through the auditory canal and through a debrided perforation of the tympanic membrane. The support ring 206 is retracted and the struts 210 or 256 are deployed to expand or open up the patch. It is also possible that the struts 210 or 256 may be constructed of a memory material such as nitinol or a polymer that will open naturally to a perpendicular position once the ring 206 is withdrawn or removed to expand the patch. The patch is aligned with the periphery of the perforation in order for the adhesive material to adhere to the underside of the tympanic membrane. Once the provider has determined that the patch is in place, the struts 210 or 256 may be retracted into the tube 202 or 252, respectively. Alternatively, the struts 210 or 256 may be retracted prior to alignment of the patch with the periphery of the perforation. When the adhesive material is sufficiently dry, the bristles 218 or 262 may be retracted into the tube 202 or 252, respectively, leaving the patch in place with a small perforation where the bristles 218 or 262 were. The device 200 or 250 may then be removed from the ear and the auditory canal and the procedure is completed. Alternatively, the adhesive material may be applied to one or more contact points between the proximal side of the patch and the debrided perforation at this time. In some situations, it may be determined that it will be necessary to bandage the ear after removal of the device 200 or 250.

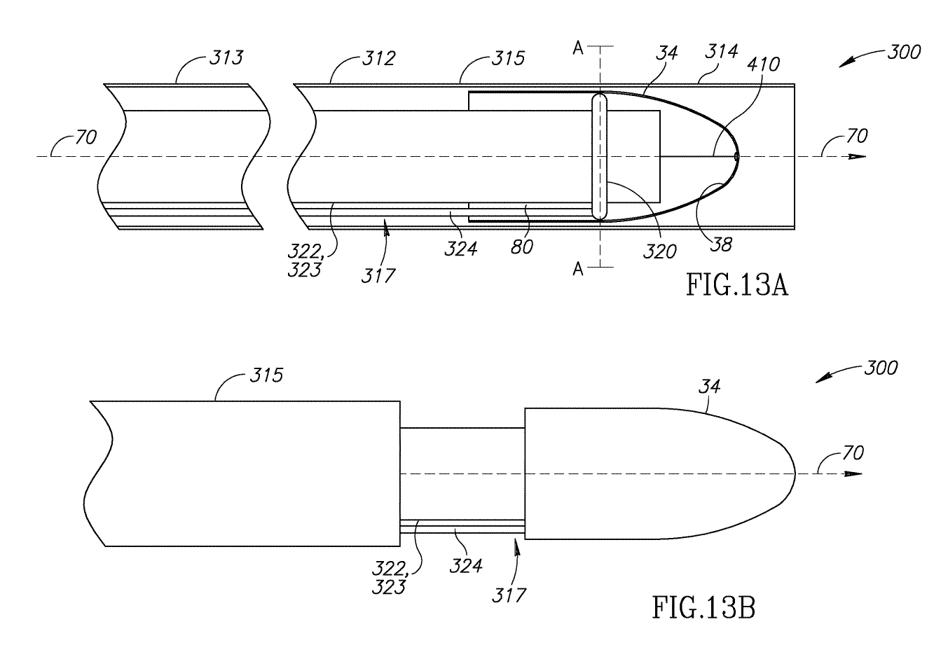

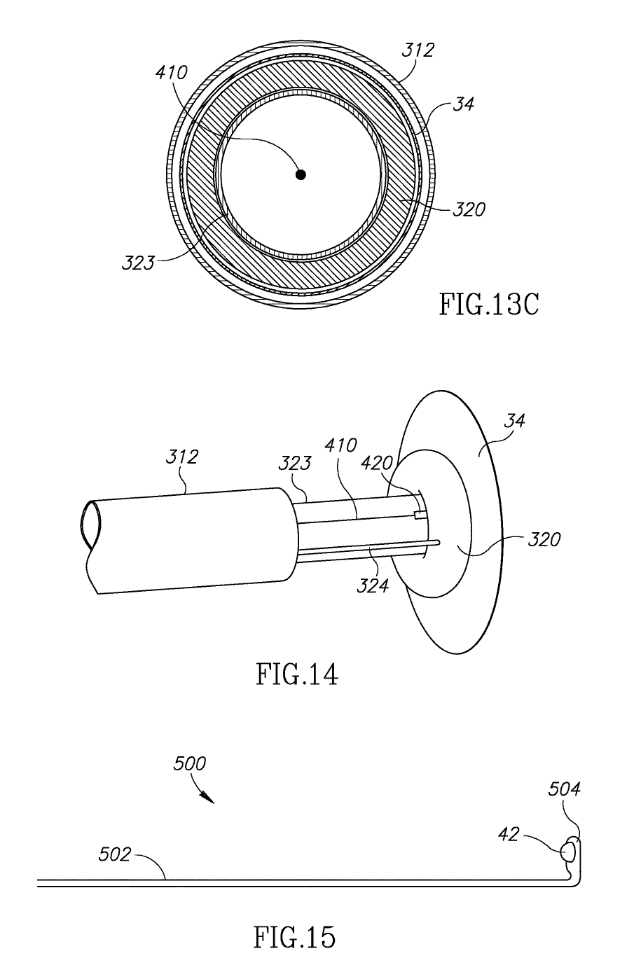

Reference is now made to FIGS. 13A and 13B, which are perspective illustrations of a tympanic membrane repair device 300 constructed in accordance with additional embodiments of the present invention. The device 300 includes an outer tube 312 having an outer tube proximal end 313, an outer tube distal end 314, and an outer tube wall 315 extending from an outer tube proximal end 313 to an outer tube distal end 314 along a longitudinal axis 70. The device 300 further includes an inner mechanism 317 which is movable in the distal/proximal directions along the longitudinal axis 70. The inner mechanism 317 includes a patch expansion mechanism 80 for expanding a patch 34 placed thereon, as will be described further hereinbelow. In the embodiment shown in FIGS. 13A and 13B, the patch expansion mechanism 80 includes an expandable balloon 320. In other embodiments, the patch expansion mechanism 80 may be a mechanical mechanism, such as described above with respect to the various embodiments shown in FIGS. 1-12. The balloon 320 may be positioned on a sliding mechanism 322, such as, for example, an inner tube 323 positioned coaxially inside of the outer tube 312 and movable with respect thereto. In some embodiments, the inner tube 323 is hollow. In other embodiments, the inner tube 323 is solid. In other embodiments, the sliding mechanism 322 is a different mechanism, such as a wire, for example. The balloon 320 may have a toroidal shape which, when inflated, is substantially perpendicular to the longitudinal axis 70, such as depicted in FIG. 14. Alternatively, the balloon 320 may have other shapes such as a disk, for example. A balloon inflation channel 324 extends through an inner tube 323 and is in fluid communication with the balloon 320 and may be incorporated into the inner tube 323, as is known in the art. In some embodiments, the inflation channel 324 is external to the inner tube 323, as shown in FIG. 13A. In some embodiments, the balloon 320 is a non-compliant balloon such that when fluid (i.e. liquid or gas) is introduced through the balloon inflation channel 324, the balloon 320 is configured to expand to its pre-determined shape. In some embodiments, the balloon 320 is a compliant balloon or a semi-compliant balloon. The patch 34 may be positioned on the balloon 320 in a collapsed state and be held in place by an outer tube 312. The patch 34 may further include a patch-flattening mechanism such as, for example, a cord 410 attached to a center of the proximal surface 38 of the patch 34 and configured to extend proximally to a proximal end of the device 300. The cord 410 is depicted as continuing proximally through the inner tube 323. The cord 410 may be used after expansion of the patch 34 to pull the patch 34 proximally until adhesion is achieved. The cord 410 may be a wire, thread, string, fiber, or similar component. Further, the outer tube 312 and/or the inner tube 323 may be hollow to house various other mechanisms or components. In some embodiments, a cord enclosure 420 may be included on the outer tube 312 or the inner tube 323, as will be described hereinbelow.

In FIGS. 13A and 13B, the tympanic membrane repair device 300 is shown in a collapsed or retracted position. In FIG. 13A, the tympanic membrane repair device 300 is shown with the inner mechanism 317 positioned within an outer tube 312. The inner mechanism 317 may be advanced through the outer tube 312 until the patch 34 is positioned distal to the outer tube 312, as shown in FIG. 13B. Once the inner mechanism 317 is positioned past the distal end 314 of the outer tube 312, the balloon 320 may be expanded, thus unfolding the patch 34, as will be described.

Reference is now made to FIG. 13C, which is a cross-sectional illustration of a distal end of the device 300. The cross-section is taken at lines A-A in FIG. 13A. The outer tube 312 is arranged coaxially with respect to the inner tube 323. The inner tube 323 has balloon 320 disposed thereon, and a patch 34 surrounds balloon 320. A cord 410 is visible in the middle of the cross section, as it runs through the inner tube 323 in the embodiment shown herein.

Reference is now made to FIG. 14, which is a schematic illustration showing the balloon 320 in an expanded state, wherein the patch 34 is also expanded and is positioned distal to the balloon 320. The cord 410 is shown hanging through an opening in the toroidal shaped balloon 320 and may extend over the inner tube 323 (as shown in FIG. 14) or through the inner tube 323 (as shown in FIG. 13C). Once the patch 34 is in place, the balloon 320 is retracted, and the cord 410 may be removed as well. The cord 410 may be removed, for example, by snipping. Alternatively, the cord 410 may be biodegradable or may deteriorate and fall off on its own. The device 300 may then be removed from the ear canal. Alternatively, the cord 410 may be removed by means of a micro-scissor or similar instrument after the device is retracted.

In some embodiments, the device 300 may further include a cord enclosure 420. The cord enclosure 420 may be included on any of the tubes, such as on the inner tube 323, as shown in FIG. 14. The cord enclosure 420 is designed to ensure that when the cord 410 is pulled proximally after the patch 34 is in place, the patch 34 does not protrude proximally through the opening of the tympanic membrane. This potentially may occur due to the relative flexibility of the patch 34 and the pressure of the taut cord 410, and the cord enclosure 420 is designed to prevent this from occurring. The cord enclosure 420 may include, for example, a ratcheting mechanism, which can be set according to settings depending on, for example, the size of the patch 34. Thus, the cord 410 may be pulled proximally by the length selected by the provider, which may be on the order of millimeters. In some embodiments, the cord enclosure 420 may further include an attachment piece for attaching a distal end of the cord 410 onto the proximal surface 38 of the patch 34, in cases in which the cord 410 is not already included on the patch 34.

In other embodiments of the present invention, the inner mechanism 317 does not include an expandable balloon. Rather, the inner mechanism 317 employs mechanical means for expanding the patch 34. The mechanical means may be, for example, a patch expansion mechanism 80, as described above with respect to the embodiments shown in FIGS. 1-12, but wherein the patch expansion mechanism 80 is inside of an outer tube 312 rather than outside of a tube 12 and wherein the outer tube 312 has an opening for advancing the patch expansion mechanism 80 distally with respect to the tube 312, after which the patch 34 is deployed. In yet additional embodiments of the present invention, the patch expansion mechanism 80 may include an expandable balloon such as the balloon 320 positioned external to the tube 12 rather than on an inner tube, wherein a patch 34 may be crimped onto the balloon 320 on the outside of the tube 12.

In some embodiments, a separate adhesive applicator 500 is contemplated. An adhesive applicator 500 is shown in one embodiment in FIG. 15. The applicator 500 may include, for example, an elongated member 502 and a hook 504 at a distal end thereof. The hook 504 may be, for example, an L-shaped surgical hook that is passed through the auditory canal with adhesive material on the hook portion. Adhesive material 42 is placed on hook 54 and can be applied at one or more of several locations around the perforation.

In some embodiments, there is provided a kit, including a repair device with a patch attached thereon, a debridement curette (disposable/reusable), adhesive material, such as fibrin glue, for example, and optionally an adhesive applicator. In embodiments of the present invention, the tympanic membrane repair device 10, 100, 200, 250, 300 or other embodiments thereof will be sized appropriately for insertion through an ear canal and for deployment of a patch on the underside of the tympanic membrane. Any suitable dimensions may be used. By ways of example only, the dimensions of the outer diameter of the device may be in a range of 1-8 mm, and in some embodiments may be approximately 2-6 mm and in some embodiments may be approximately 4 mm. The patch 34 may also have a suitable range of diameters, for example, 2-6 mm, or in some embodiments approximately 4-5 mm. The patch 34 should be configured to fully cover the opening in the tympanic membrane and enable it to be placed over and optionally glued onto the debrided perforation.

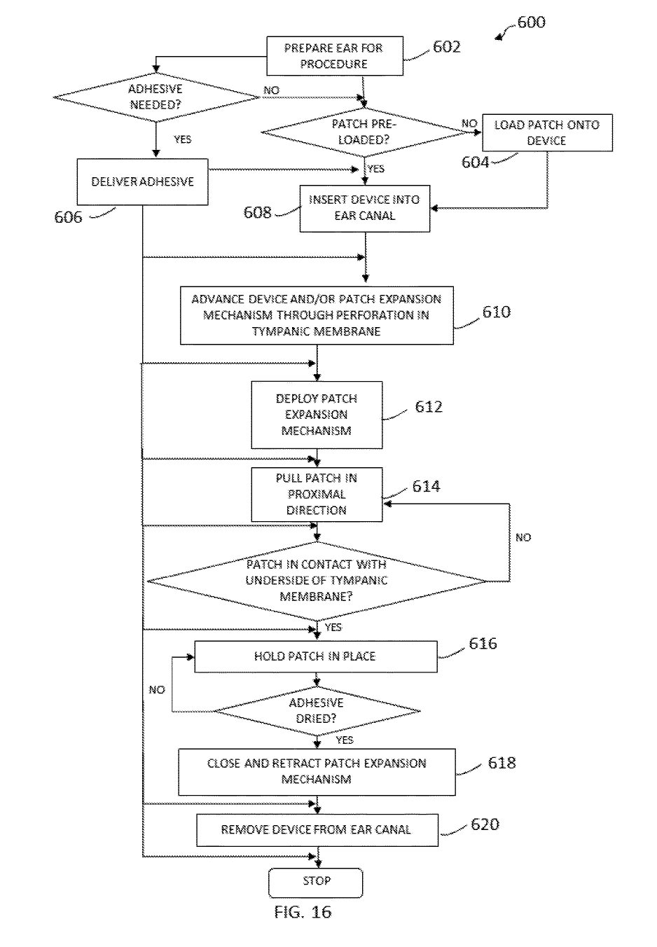

Reference is now made to FIG. 16, which is a flow chart diagram depicting the steps of a method 600 of repairing an eardrum using tympanic membrane repair device 10, 100, 200, 250, 300 or similar embodiments, in accordance with embodiments of the present invention. First, a provider or other medical professional prepares the ear (step 602) for the procedure. The following steps may be included in the preparation: First, the practitioner may anesthetize the ear. Then the practitioner may examine the perforation in the tympanic membrane in order to determine whether it is suitable to enable the practitioner to inspect the middle ear of the patient for disease or injury. If the provider determines that the perforation is not suitable for such an inspection, then the provider may create a flap by making an incision on the periphery of the tympanic membrane with a curette, for example, and pulling up the incised tissue with a forceps, for example, so that the endoscope can pass through the slit created thereby. Alternatively or in addition, the provider may introduce an otoscope, endoscope, microscope or other instrument into the ear canal to the opening in the tympanic membrane. The provider may then use the curette to debride the perforation to a desired shape and size. In some embodiments, the same curette is used but if necessary, the curette may be switched for a more suitable instrument. In some embodiments, the patch 34 is included on the repair device as a unit. In other embodiments, the repair device is selected by the provider, and the patch 34 is then loaded (step 604) onto or into the repair device before inserting the repair device into the ear canal. This can be done, for example, in the provider's office.

In some embodiments, adhesive material is needed and may be delivered (step 606) at various stages, such as before or after the patch is loaded onto the device (step 604), after the device is inserted into the ear canal (step 608), after the patch is pulled proximally (step 614) after removal of the device (step 620) or potentially at other steps along the way. In some embodiments, no adhesive material is required to be applied at the time of the procedure due to the nature of the composition of the patch 34, as disclosed supra. In some embodiments, adding adhesive material may be done using a separate adhesive applicator 500, as described above with respect to FIG. 15. In other embodiments, the adhesive material may be delivered through the repair device after the device is inserted into the ear canal. Alternatively, the adhesive material may be delivered through the ear canal after the device is withdrawn. The adhesive material may be delivered to the inside of the tympanic membrane, or to the proximal surface of the patch 34, or to points of contact between the proximal side of the patch and the debrided perforation. In some embodiments, several small dabs of adhesive material are applied around the circumference of the area to which the patch will be applied. In yet additional embodiments, a two-stage adhesive process is employed, whereby a first adhesive component is applied, the position of the patch 34 is checked, and if necessary, adjusted, and then a second adhesive component is applied. Only after the second adhesive component is applied does the adhesive quality take effect. This enables the provider to ensure that placement is correct before adhesion occurs. In some embodiments, an additional lumen may be included in the device to provide a compartment for the second adhesive component. In yet additional embodiments, no adhesive material is used at all, and the patch 34 stays in place due to its being in contact with the debrided vascular wall.

Next, the repair device is inserted (step 608) into the ear canal. Next, either the entire device or just the patch expansion mechanism 80 is advanced (step 610) through the perforation in the tympanic membrane. For example, in the embodiment shown wherein an inner mechanism 317 is used (for example, FIGS. 13A and 13B), the inner mechanism 317 may be advanced past the distal end of the outer tube 312 and through the opening of the tympanic membrane. In other embodiments, after the entire device is advanced, the inner mechanism is advanced through the opening. Next, the patch expansion mechanism 80 is deployed (step 612), thus expanding the patch into its flattened shape. Next, the patch 34 is pulled proximally (step 614) against the tympanic membrane, and held in place (step 616) until the adhesive material has dried. In some embodiments, this is done via a cord 410 or other patch-flattening mechanism. In other embodiments, this is done by a distal patch holder 90 such as a hook or bristles. When the patch 34 is securely in place, the patch expansion mechanism is closed and retracted (step 618) and the repair device is removed (step 620) from the ear canal. Generally, epithelial cells will grow over the patch, causing the perforation in the tympanic membrane to heal with minimal conductive hearing loss.

From all that has been said, it will be clear that there has thus been shown and described herein a tympanic membrane repair device which fulfills the various advantages sought therefor.

It will become apparent to those skilled in the art, however, that many changes, modifications, variations, and other uses and applications of the subject tympanic membrane repair device are possible and contemplated. All changes, modifications, variations, and other uses and applications which do not depart from the spirit and scope of the disclosure are deemed to be covered by the disclosure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

While certain features of the present invention have been illustrated and described herein, many modifications, substitutions, changes, and equivalents may occur to those of ordinary skill in the art. It is, therefore, to be understood that the appended claims are intended to cover all such modifications and changes as fall within the true spirit of the present invention.

1. A tympanic membrane repair device, the device comprising:
a tube having a tube proximal end, a tube distal end and a tube wall extending from said tube proximal end to said tube distal end along a longitudinal axis; and
a patch expansion mechanism adjacent to said tube, said patch expansion mechanism having a retracted configuration and an expanded configuration, wherein in said retracted configuration said patch expansion mechanism is positioned in close proximity to said tube wall and wherein in said expanded configuration said patch expansion mechanism is configured to at least partially extend away from said tube wall at an angle to said longitudinal axis.

2. The device of claim 1, wherein said patch expansion mechanism is positioned external to said tube wall.

3. The device of claim 1, wherein said patch expansion mechanism is positioned internal to said tube wall.

4. The device of claim 1, wherein said patch expansion mechanism comprises:
a sliding mechanism configured to move in a proximal and distal direction along said longitudinal axis;
a patch expansion member having an expansion member proximal end and an expansion member distal end, wherein in said retracted configuration, said expansion member proximal end is held by said sliding mechanism such that said patch expansion member is substantially aligned with said longitudinal axis, and wherein upon movement of said sliding mechanism, said patch expansion member is configured to extend outwardly from said tube wall distal end.

5. The device of claim 4, wherein said sliding mechanism comprises:
a patch support ring surrounding said patch expansion member; and
a patch support ring retraction mechanism attached to said patch support ring, wherein said movement of said sliding mechanism comprises proximal movement of said patch support ring retraction mechanism resulting in release of said patch expansion member from said patch support ring.

6. The device of claim 4, wherein said sliding mechanism comprises:
a runner positioned coaxially with respect to said tube wall, said expansion member proximal end attached to said runner; and
a stop member at said tube distal end, wherein said movement of said sliding mechanism comprises distal movement of said runner until said runner reaches said stop member, resulting in distal and outwardly extending movement of said patch expansion member proximal end.

7. The device of claim 1, further comprising a distal patch holder at said tube distal end.

8. The device of claim 7, wherein said distal patch holder is at least one of: a curved hook, a straight hook, or a set of bristles held by a removable cap.

9. The device of claim 1, wherein said patch expansion mechanism comprises an expandable balloon positioned such that when in an expanded state, said expandable balloon is substantially perpendicular to said tube.

10. The device of claim 9, wherein said expandable balloon is a toroidal shaped balloon.

11. The device of claim 9, wherein said tube distal end has an opening, said patch expansion mechanism further comprising a sliding mechanism attached to said expandable balloon, wherein said sliding mechanism is configured to push said expandable balloon distally until said expandable balloon exits said tube through said opening.

12. The device of claim 9, further comprising a balloon inflation channel extending from said balloon to said tube proximal end, said balloon inflation channel configured to provide inflation fluid into said expandable balloon.

13. The device of claim 1, further comprising a repair patch having a flexible surface with outer edges, said repair patch held by said patch expansion mechanism, wherein in said retracted configuration, said flexible surface is folded into an umbrella-like shape wherein said outer edges are in close proximity to said tube wall and in said expanded configuration, said flexible surface assumes a substantially flat configuration wherein said outer edges are extended away from said tube wall.

14. The device of claim 13, further comprising a cord positioned at a center of said repair patch on an inner side of said flexible surface.

15. The device of claim 1, wherein the tube further comprises an input port connected to various output ports through an interior lumen formed in the tube.

16. A method of repairing a perforation in a tympanic membrane of an ear, the method comprising:
providing a repair device comprising a tube having a tube proximal end, a tube distal end, and a tube wall extending from said tube proximal end to said tube distal end along a longitudinal axis and a patch expansion mechanism having a retracted configuration and an expanded configuration;
providing a repair patch held by said patch expansion mechanism in said retracted configuration, such that outer edges of said repair patch are in close proximity to said tube wall;
inserting the repair device into an ear canal and through a perforation in a tympanic membrane of the ear;
expanding the patch by operation of the patch expansion mechanism, such that said outer edges of said repair patch are extended away from said tube wall;
adhering the patch to an underside of the tympanic membrane;
retracting the patch expansion mechanism; and
removing the repair device from the ear canal.

17. The method of claim 16, further comprising inserting an adhesive material into the ear canal for adhering said repair patch to the tympanic membrane.

18. The method of claim 17, wherein said inserting an adhesive material is done through an interior lumen of said tube.

19. The method of claim 17, wherein said inserting an adhesive material is done using a separate adhesive applicator.

20. The method of claim 16, wherein said expanding comprises expanding a balloon.
